(19) United States
(12) Patent Application Publication
(10) Pub. No.: US 2023/0263990 A1
(43) Pub. Date: Aug. 24, 2023

(54) SEAMLESS EXPANDABLE INTRODUCER SHEATH

(71) Applicant: BOSTON SCIENTIFIC SCIMED, INC., MAPLE GROVE, MN (US)

(72) Inventors: Tim O'Connor, Galway (IE); Sean Shanley, Galway (IE); Richard O'Sullivan, Turloughmore (IE); Neil O'Connor, Oranmore (IE)

(73) Assignee: BOSTON SCIENTIFIC SCIMED, INC., MAPLE GROVE, MN (US)

(21) Appl. No.: 18/112,018

(22) Filed: Feb. 21, 2023

Related U.S. Application Data
(60) Provisional application No. 63/312,464, filed on Feb. 22, 2022.

(51) Int. Cl. A61M 25/00

(52) U.S. Cl. CPC A61M 25/0023; A61M 25/005; A61M 2025/0024

(57) ABSTRACT

An introducer sheath assembly includes an expandable introducer sheath including an expandable braid defining an elongate tubular member moveable between a collapsed configuration for delivery and an expanded configuration for deployment, the expandable braid having a first length when in the collapsed configuration and a second, reduced, length when in the expanded configuration. The introducer sheath assembly includes a dilator adapted to releasably hold the expandable introducer sheath in its collapsed configuration for delivery until the dilator is actuated to release the expandable braid from the dilator, thereby permitting the expandable braid to expand to its expanded configuration.

### CROSS-REFERENCE TO RELATED APPLICATIONS

This application claims the benefit of priority of U.S. Provisional Application No. 63/312,464, filed Feb. 22, 2022, the entire disclosure of which is hereby incorporated by reference.

### TECHNICAL FIELD

The present disclosure pertains to medical devices, and methods for manufacturing and using medical devices. More particularly, the disclosure is directed to an expandable introducer sheath.

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### SUMMARY

This disclosure provides design, material, manufacturing method, and use alternatives for medical devices. As an example, an introducer sheath assembly includes an expandable introducer sheath and a dilator. The expandable introducer sheath includes an expandable braid moveable between a collapsed configuration for delivery and an expanded configuration for deployment, the expandable braid having a first length when in the collapsed configuration and a second, reduced, length when in the expanded configuration. The dilator is adapted to releasably hold the expandable introducer sheath in its collapsed configuration for delivery until the dilator is actuated to release the expandable braid from the dilator, thereby permitting the expandable braid to expand to its expanded configuration.

Alternatively or additionally, the expandable introducer sheath may further include an adhesive band disposed on the dilator, the adhesive band adapted to engage the expandable introducer sheath until axial movement of the dilator relative to the expandable introducer sheath causes the adhesive band to disengage the expandable introducer sheath.

Alternatively or additionally, the dilator may further include one or more securement features and the expandable braid may further include one or more securement elements adapted to releasably engage the one or more securement features, and to disengage with the one or more securement features in response to axial movement of the dilator relative to the expandable introducer sheath.

Alternatively or additionally, the one or more securement features may include one or more slits formed in a distal end of the dilator.

Alternatively or additionally, the one or more securement features may include one or more tabs disposed on an outer surface of the dilator.

Alternatively or additionally, the one or more securement elements may include one or more loops formed within an end of the expandable braid.

Alternatively or additionally, the expandable braid may include a Nitinol braid.

Alternatively or additionally, the expandable introducer sheath assembly may further include a polymeric covering disposed relative to the expandable braid.

Alternatively or additionally, the polymeric covering may include a spray coated polyurethane or a spray coated silicone.

Alternatively or additionally, the expandable introducer sheath assembly may further include one or more radiopaque markers secured relative to the expandable braid.

As another example, an expandable introducer sheath includes an expandable braid moveable between a collapsed configuration for delivery and an expanded configuration for deployment, and a polymeric covering extending over the expandable braid, the polymeric covering adapted to accommodate the expandable braid moving between its collapsed configuration and its expanded configuration.

Alternatively or additionally, the expandable braid may be moveable between its collapsed configuration and its expanded configuration without utilizing any folded or foldable seams.

Alternatively or additionally, the expandable braid may have a first length when in the collapsed configuration and a second, reduced, length when in the expanded configuration.

Alternatively or additionally, the expandable braid may be biased into its expanded configuration.

Alternatively or additionally, the expandable braid may be adapted to move into its collapsed configuration when the expandable braid is elongated.

Alternatively or additionally, the expandable braid may be adapted to be deployed using a dilator that includes one or more engagement features adapted to releasably couple the dilator to the expandable braid.

As another example, a medical device includes an expandable braid biased into an expanded configuration for deployment, wherein elongating the expandable braid causes the expandable braid to move into an elongated configuration in which the expandable braid is longer and narrower than when in its expanded configuration, and a dilator that is adapted to releasably hold the expandable braid in its elongated configuration.

Alternatively or additionally, the medical device may further include an adhesive band disposed on the dilator, the adhesive band adapted to engage the expandable braid until axial movement of the dilator relative to the expandable braid causes the adhesive band to disengage the expandable braid.

Alternatively or additionally, the dilator may further include one or more securement features and the expandable braid may further include one or more securement elements adapted to releasably engage the one or more securement features and to disengage with the one or more securement features in response to axial movement of the dilator relative to the expandable braid.

Alternatively or additionally, the medical device may further include a polymeric covering extending over the expandable braid.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of an illustrative braided introducer sheath shown coupled with a dilator, with the braided introducer sheath shown in a delivery configuration;

FIG. 2 is a perspective view of the illustrative braided introducer sheath of FIG. 1, shown with the braided introducer sheath shown uncoupled from the dilator in a deployment configuration;

FIG. 3 is a perspective view of an illustrative braided introducer sheath shown coupled with a dilator, with the braided introducer sheath shown in a delivery configuration;

FIG. 4 is a perspective view of the illustrative braided introducer sheath of FIG. 3, shown with the braided introducer sheath shown uncoupled from the dilator in a deployment configuration;

FIG. 5 is a schematic side view of an illustrative braided introducer sheath including loops for releasably securing the illustrative braided introducer sheath to a dilator;

FIG. 6 is a schematic side view of an illustrative dilator usable with the illustrative braided introducer sheath of FIG. 5;

FIG. 7 is a perspective view of an illustrative braided introducer sheath; and

FIGS. 8A and 8B are schematic cross-sectional views taken along line 8-8 of FIG. 7.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Introducer sheaths are used for introducing a variety of different medical devices into a patient's vasculature. Some introducer sheathes are expandable, meaning that they can be advanced into the vasculature in a collapsed or compressed configuration prior to being expanded in order to allow particular medical devices to pass through them. Some expandable introducer sheathes include one, two, three or more folded seams. When the one or more folded seams remain folded, the expandable introducer sheath may be considered as being in a compressed or collapsed configuration for delivery. Unfolding the one or more folded seams means that the expandable introducer sheath expands as the one or more folded seams unfold. In some cases, an expandable introducer sheath having one or more folded seams expands (as the one or more folded seams unfold) in response to a medical device being passed through the expandable introducer sheath that has one or more dimensions sufficient to cause expansion of the expandable introducer sheath. In some instances, an inflatable balloon may be advanced into the expandable introducer sheath (with the one or more folded seams intact) and the inflatable balloon may be inflated in order to cause the one or more folded seams to unfold, thereby causing the expandable introducer sheath to expand. It will be appreciated that there can be difficulties in having multiple folded seams all opening symmetrically, and in some cases, folded seams may be relatively stiff.

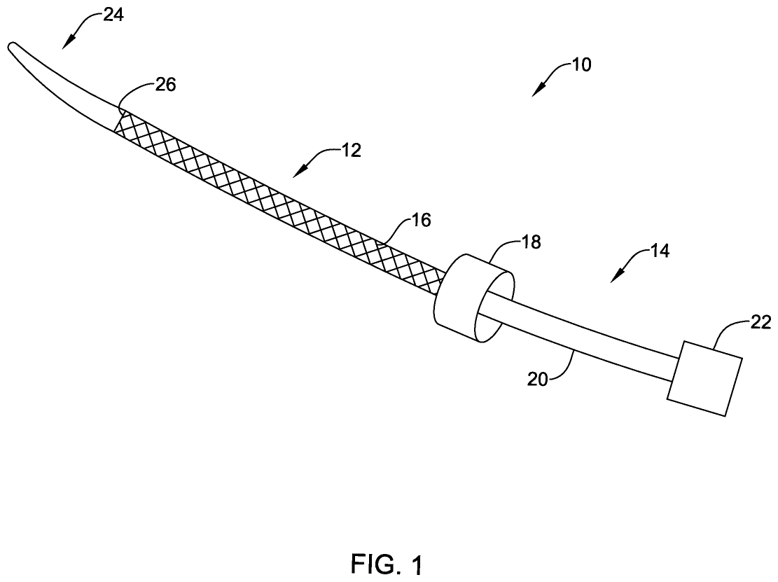

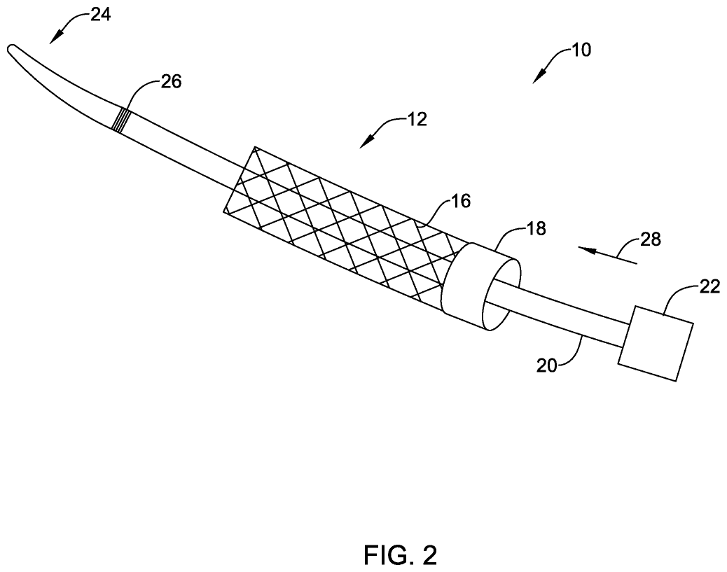

FIG. 1 and FIG. 2 are perspective views of an illustrative expandable introducer sheath assembly 10. The illustrative expandable introducer sheath assembly 10 includes an expandable introducer sheath 12 and a dilator 14 that may be used to advance the expandable introducer sheath 12 through the vasculature while retaining the expandable introducer sheath 12 in a delivery configuration, as will be discussed. FIG. 1 shows the expandable introducer sheath 12 in a collapsed configuration for delivery while FIG. 2 shows the expandable introducer sheath 12 in an expanded configuration for deployment or use.

The expandable introducer sheath 12 includes an expandable braid 16 that is moveable between a collapsed configuration for delivery and an expanded configuration for deployment, the expandable braid having a first length when in the collapsed configuration and a second, reduced, length when in the expanded configuration without requiring or relying upon any folded seams in order to move from the collapsed configuration to the expanded configuration. The expandable braid 16 may be formed of any desired material. In some cases, the expandable braid 16 may be formed of a shape memory material that is adapted to learn a “remembered” position even after being deformed from that “remembered” position. In some cases, the expanded configuration corresponds to the “remembered” position. The expandable braid 16 may be formed of a shape memory polymer, for example. In some cases, the expandable braid 16 may be formed of a shape memory metal, such as but not limited to the nickel-titanium alloys including Nitinol.

The expandable introducer sheath 12 also includes a proximal end 18 that is adapted to allow the dilator 14 to extend through the expandable introducer sheath 12. In some instances, the proximal end 18 may be adapted to allow other devices to be passed through the expandable introducer sheath 12. As seen, the proximal end 18 may have an overall diameter that equals or at least substantially equals a diameter of the expandable braid 16 when the expandable braid 16 is in its expanded, shorter length. In some instances, the proximal end 18 may include a hemostasis valve that may be adhesively bonded, laser welded or insert molded to the expandable braid 16.

The dilator 14 includes an elongate body 20 extending from a handle 22 to an atraumatic tip 24. The atraumatic tip 24 may be straight or curved, for example, and may be adapted for advancement through the patient's vasculature. The atraumatic tip 24 may be stiff or soft, depending on the specific vasculature through which the expandable introducer sheath assembly 10 will be advanced. The handle 22 may be used to move the dilator 14 relative to the expandable introducer sheath 12. The handle 22 may take any desired form, for example. The dilator 14 may be formed of any suitable polymeric material, for example. In some instances, the dilator 14 may be formed of a low friction flexible polymeric material such as but not limited to LDPE (low density polyethylene), HDPE (high density polyethylene), polypropylene and Pebax.

While the expandable braid 16 is illustrated as having a cylindrical profile, with a circular cross-section, this is not required. The expandable braid 16 may have any desired cross-sectional shape, such as but not limited to a rectilinear cross-sectional shape, a triangular cross-sectional shape or a polygonal cross-sectional shape. Similarly, while the dilator 14 is shown as having a circular cross-sectional shape, this is not required. In some cases, the dilator 14 may have any desired cross-sectional shape, such as but not limited to a rectilinear cross-sectional shape, a triangular cross-sectional shape or a polygonal cross-sectional shape.

The expandable introducer sheath assembly 10 can include one or more features that allows the dilator 14 to hold the expandable braid 16 in its collapsed, longer length, configuration as shown in FIG. 1. In some cases, the expandable introducer sheath assembly 10 includes an adhesive band 26, a portion of which can be seen peeking out from under the expandable introducer sheath 12 in FIG. 1. The adhesive band 26 has a width that exceeds that shown in FIG. 1, as can be seen in FIG. 2. The adhesive band 26 may be adapted to lightly secure the expandable introducer sheath 12 relative to the dilator 14 with sufficient adhesiveness to allow the expandable introducer sheath assembly 10 to be advanced through the vasculature yet allowing relative axial movement between the expandable introducer sheath 12 and the dilator 14 to break this adhesion and allow the adhesive band 26 to let go of the expandable introducer sheath 12. A variety of different adhesives may be used in forming the adhesive band 26. Examples include but are not limited to RTV (room temperature vulcanizing) silicone, low durometer polyurethane adhesive, and neoprene adhesive.

In some cases, once a desired location within the patient's vasculature has been reached, a user may cause the expandable introducer sheath 12 to move from its collapsed, delivery configuration to an expanded, deployment configuration by causing the dilator 14 to move axially relative to the expandable introducer sheath 12. In some cases, as shown in FIG. 2, the user may move the dilator 14 axially relative to the expandable introducer sheath 12 by moving the dilator 14 in a direction indicated by an arrow 28. As the dilator 14 moves distally relative to the expandable introducer sheath 12, the added force overcomes the attractive force provided by the adhesive band 26, and the expandable introducer sheath 12 is permitted to regain its “remembered” shape, i.e., the expanded configuration shown in FIG. 2. Once the expandable introducer sheath 12 has broken free of the adhesive band 26, the dilator 14 may be withdrawn from the expandable introducer sheath 12.

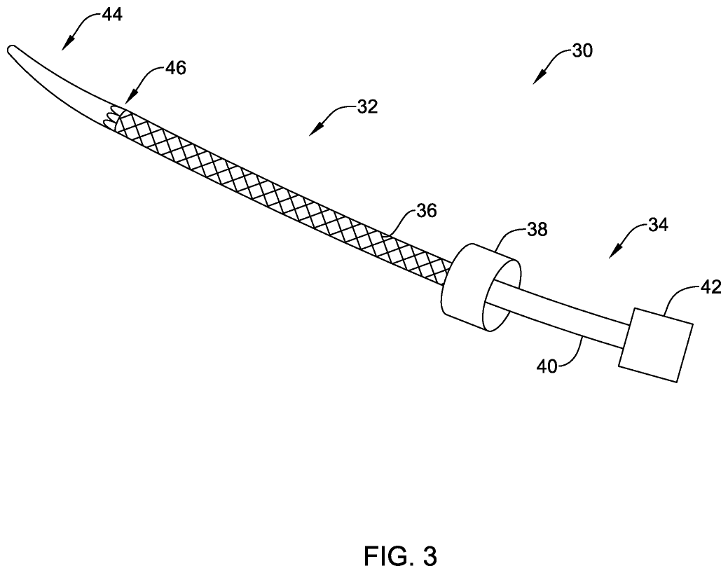

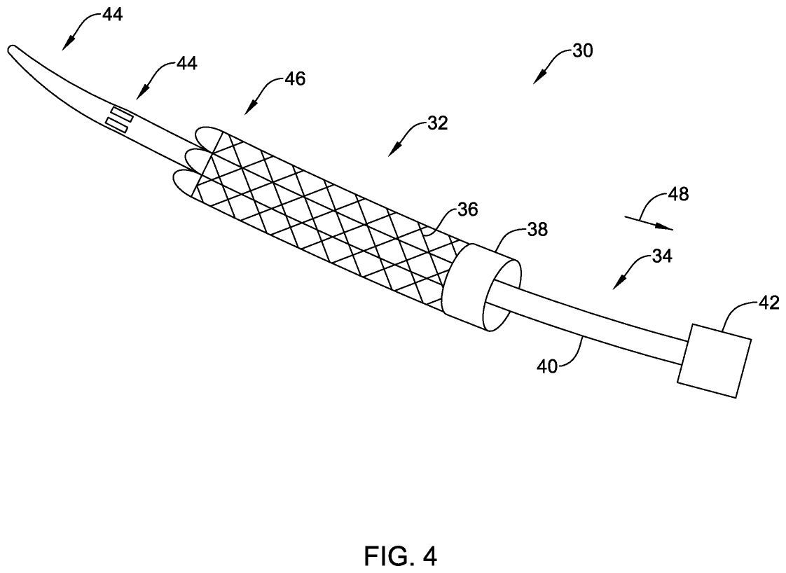

FIG. 3 and FIG. 4 are perspective views of an illustrative expandable introducer sheath assembly 30. The illustrative expandable introducer sheath assembly 30 includes an expandable introducer sheath 32 and a dilator 34 that may be used to advance the expandable introducer sheath 32 through the vasculature while retaining the expandable introducer sheath 32 in a delivery configuration, as will be discussed. FIG. 3 shows the expandable introducer sheath 32 in a collapsed configuration for delivery while FIG. 4 shows the expandable introducer sheath 32 in an expanded configuration for deployment or use.

The expandable introducer sheath 32 includes an expandable braid 36 that is moveable between a collapsed configuration for delivery and an expanded configuration for deployment, the expandable braid having a first length when in the collapsed configuration and a second, reduced, length when in the expanded configuration without requiring or relying upon any folded seams in order to move from the collapsed configuration to the expanded configuration. The expandable braid 36 may be formed of any desired material. In some cases, the expandable braid 36 may be formed of a shape memory material that is adapted to learn a “remembered” position even after being deformed from that “remembered” position. In some cases, the expanded configuration corresponds to the “remembered” position. The expandable braid 36 may be formed of a shape memory polymer, for example. In some cases, the expandable braid 36 may be formed of a shape memory metal, such as but not limited to the nickel-titanium alloys including Nitinol.

The expandable introducer sheath 32 also includes a proximal end 38 that is adapted to allow the dilator 34 to extend through the expandable introducer sheath 32. In some instances, the proximal end 38 may be adapted to allow other devices to be passed through the expandable introducer sheath 32. As seen, the proximal end 38 may have an overall diameter that equals or at least substantially equals a diameter of the expandable braid 36 when the expandable braid 36 is in its expanded, shorter length, configuration.

The dilator 34 includes an elongate body 40 extending from a handle 42 to an atraumatic tip 44. The atraumatic tip 44 may be straight or curved, for example, and may be adapted for advancement through the patient's vasculature. The atraumatic tip 44 may be stiff or soft, depending on the specific vasculature through which the expandable introducer sheath assembly 30 will be advanced. The handle 42 may be used to move the dilator 34 relative to the expandable introducer sheath 32. The handle 42 may take any desired form, for example. The dilator 34 may be formed of any suitable polymeric material, for example. In some instances, the dilator 34 may be formed of a low friction flexible polymeric material such as but not limited to LDPE (low density polyethylene), HDPE (high density polyethylene), polypropylene and Pebax.

While the expandable braid 36 is illustrated as having a cylindrical profile, with a circular cross-section, this is not required. The expandable braid 36 may have any desired cross-sectional shape, such as but not limited to a rectilinear cross-sectional shape, a triangular cross-sectional shape or a polygonal cross-sectional shape. Similarly, while the dilator 34 is shown as having a circular cross-sectional shape, this is not required. In some cases, the dilator 34 may have any desired cross-sectional shape, such as but not limited to a rectilinear cross-sectional shape, a triangular cross-sectional shape or a polygonal cross-sectional shape.

The expandable introducer sheath assembly 30 can include one or more features that allows the dilator 34 to hold the expandable braid 36 in its collapsed, longer length, configuration as shown in FIG. 4. In some cases, for example, and as best seen in in FIG. 4, the dilator 34 may include one or more securement features 44. The expandable braid 36 may include one or more securement elements 46 that may be adapted to releasably engage with the one or more securement features 44 in order to secure the expandable introducer sheath 32 in its collapsed, longer length, configuration as shown in FIG. 4. In some cases, as shown, the one or more securement features 44 may include bumps or tabs formed on a surface of the dilator 34 that are adapted to releasably engage with loops formed as part of the expandable braid 36. These are just examples. The one or more securement features 44 on the dilator 34 may also include hooks that can engage loops formed as part of the expandable braid 36.

In some cases, the one or more securement features 44 on the dilator 34 and the one or more securement elements 46 on the expandable braid 36 may be disengaged by moving the dilator 34 axially in a direction, relative to the expandable introducer sheath 32, indicated by an arrow 48. Once disengaged, the expandable braid 36 may be free to revert to its “remembered” configuration, as shown in FIG. 4. Once disengaged from the expandable introducer sheath 32, the dilator 34 may be withdrawn from the expandable introducer sheath 32.

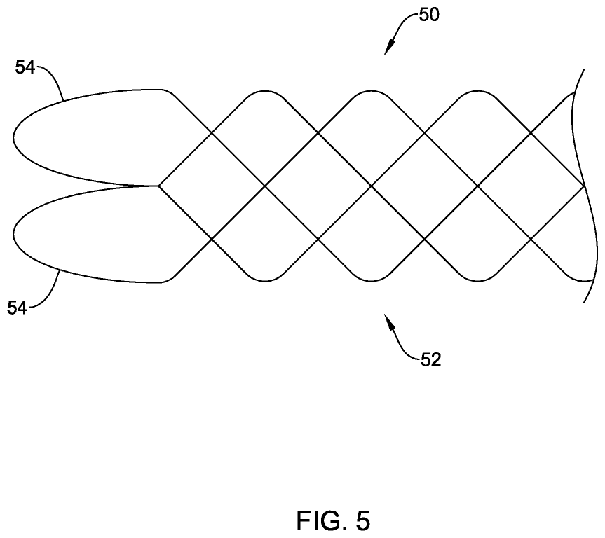

FIG. 5 is a schematic side view of an illustrative expandable introducer sheath 50. The illustrative expandable introducer sheath 50 may be considered as being an example of the expandable introducer sheath 12 and/or the expandable introducer sheath 32. The expandable introducer sheath 50 includes an elongate tubular body 52. The expandable introducer sheath 50 also includes several loops 54 that may serve as securement elements such as the one or more securement elements 44 (FIGS. 3 and 4). In some cases, the loops 54 may be integrally formed as part of the elongate tubular body 52. In some instances, the loops 54 may be separately formed and subsequently attached to the elongate tubular body 52.

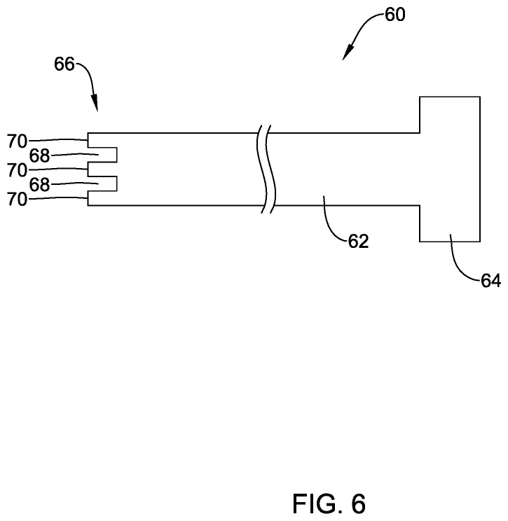

FIG. 6 is a schematic side view of an illustrative dilator 60. The illustrative dilator 60 may be considered as being an example of the dilator 14 and/or the dilator 34. The dilator 60 may be considered as being adapted for use with the expandable introducer sheath 50 (FIG. 5), for example. The dilator 60 includes an elongate body 62 extending from a handle 64 to a distal end 66. While not shown, in some cases, the elongate body 62 may extend distally beyond what is shown as the distal end 66. The distal end 66 includes several slots 68 that together define several corresponding tabs 70. It will be appreciated that the tabs 70 are adapted to easily engage with the loops 54 discussed previously with respect to the expandable introducer sheath 50 of FIG. 5. Moving the dilator 60 proximally relative to the expandable introducer sheath 50 will cause the loops 54 to disengage with the tabs 70.

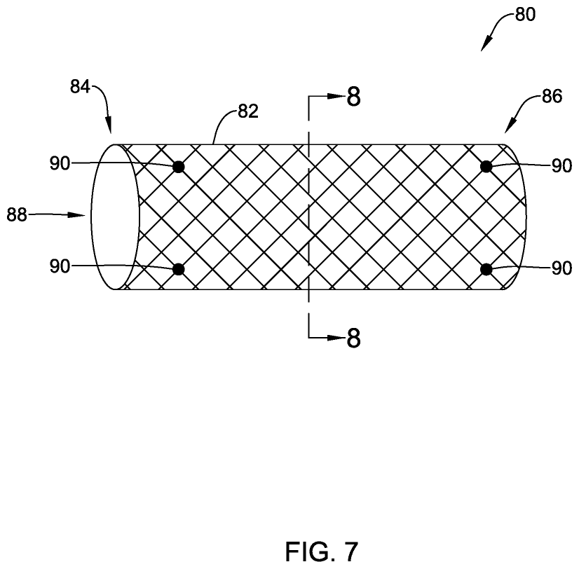

FIG. 7 is a perspective view of an illustrative braided introducer sheath 80. The illustrative braided introducer sheath 80 may be considered as being an example of the expandable introducer sheath 12, the expandable introducer sheath 32, and/or the expandable introducer sheath 50. The braided introducer sheath 80 includes an elongate tubular body 82 extending from a first end 84 to a second end 86. The elongate tubular body 82 defines a lumen 88 that extends through the elongate tubular body 82. In some cases, the braided introducer sheath 80 may include one or more radiopaque markers 90 that may be secured to the braided introducer sheath 80 in order to provide additional visibility to the braided introducer sheath 80 under fluoroscopy.

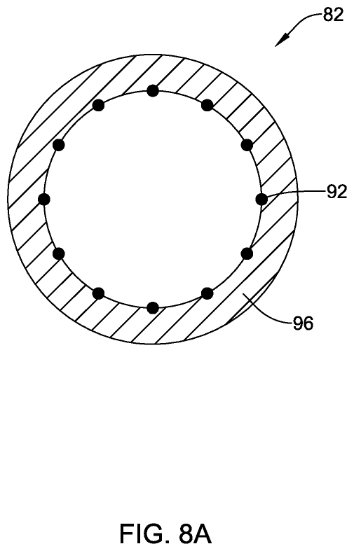

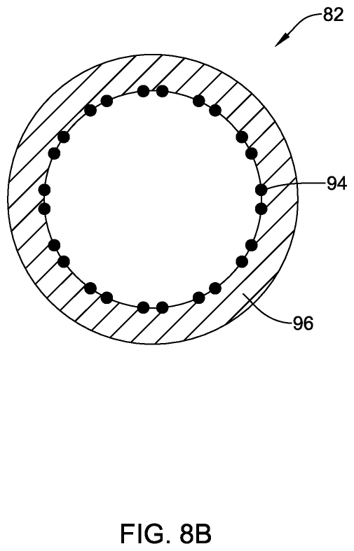

The elongate tubular body 82 is braided. The elongate tubular body 82 may be braided as a 1×1 braiding pattern, a 2×2 braiding pattern, a 3×3 braiding pattern, or any desired braiding pattern. FIG. 8A is a schematic cross-sectional view of the elongate tubular body 82, showing a braided structure 92 having a 1×1 braiding pattern while FIG. 8B is a schematic cross-sectional view of the elongate tubular body 82, showing a braided structure 94 having a 2×2 braiding pattern. These are just illustrative, as other braiding patterns are also contemplated. In some cases, knitted patterns, which are formed from a single filament, may also exhibit a similar property in that pulling on the knitted pattern axially can cause the knitted pattern to reduce in diameter and increase in length, are also contemplated.

The braided introducer sheath 80 includes a polymer covering 96. It will be appreciated that the addition of the polymer covering 96 to the braided introducer sheath 80 may mean that the braided introducer sheath 80 is fluid-impermeable, meaning that fluids can be passed through the braided introducer sheath 80 without leaking laterally through a wall of the braided introducer sheath 80. In some cases, the polymer covering 96 may include a spray-coated polyurethane or a spray-coated silicone. In some instances, the polymer covering 96 may be applied via a dip coating process, for example.

The devices described herein, as well as various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304 L, and 316 LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An introducer sheath assembly, comprising:
an expandable introducer sheath including an expandable braid moveable between a collapsed configuration for delivery and an expanded configuration for deployment, the expandable braid having a first length when in the collapsed configuration and a second, reduced, length when in the expanded configuration; and
a dilator adapted to releasably hold the expandable introducer sheath in its collapsed configuration for delivery until the dilator is actuated to release the expandable braid from the dilator, thereby permitting the expandable braid to expand to its expanded configuration.

2. The introducer sheath assembly of claim 1, further comprising an adhesive band disposed on the dilator, the adhesive band adapted to engage the expandable introducer sheath until axial movement of the dilator relative to the expandable introducer sheath causes the adhesive band to disengage the expandable introducer sheath.

3. The introducer sheath assembly of claim 1, wherein:
the dilator further comprises one or more securement features; and
the expandable braid further comprises one or more securement elements adapted to releasably engage the one or more securement features; and
the one or more securement elements are adapted to disengage with the one or more securement features in response to axial movement of the dilator relative to the expandable introducer sheath.

4. The introducer sheath assembly of claim 3, wherein the one or more securement features comprise one or more slits formed in a distal end of the dilator.

5. The introducer sheath assembly of claim 3, wherein the one or more securement features comprise one or more tabs disposed on an outer surface of the dilator.

6. The introducer sheath assembly of claim 3, wherein the one or more securement elements comprise one or more loops formed within an end of the expandable braid.

7. The introducer sheath assembly of claim 1, wherein the expandable braid comprises a Nitinol braid.

8. The introducer sheath assembly of claim 1, wherein the expandable introducer sheath further comprises a polymeric covering disposed relative to the expandable braid.

9. The introducer sheath assembly of claim 8, wherein the polymeric covering comprises a spray coated polyurethane or a spray coated silicone.

10. The introducer sheath assembly of claim 1, further comprising one or more radiopaque markers secured relative to the expandable braid.

11. An expandable introducer sheath, comprising:
an expandable braid moveable between a collapsed configuration for delivery and an expanded configuration for deployment; and
a polymeric covering extending over the expandable braid, the polymeric covering adapted to accommodate the expandable braid moving between its collapsed configuration and its expanded configuration.

12. The expandable introducer sheath of claim 11, wherein the expandable braid is moveable between its collapsed configuration and its expanded configuration without utilizing any folded or foldable seams.

13. The expandable introducer sheath of claim 11, wherein the expandable braid has a first length when in the collapsed configuration and a second, reduced, length when in the expanded configuration.

14. The expandable introducer sheath of claim 13, wherein the expandable braid is biased into its expanded configuration.

15. The expandable introducer sheath of claim 14, wherein the expandable braid is adapted to move into its collapsed configuration when the expandable braid is elongated.

16. The expandable introducer sheath of claim 12, wherein the expandable braid is adapted to be deployed using a dilator that includes one or more engagement features adapted to releasably couple the dilator to the expandable braid.

17. A medical device, comprising:
an expandable braid biased into an expanded configuration for deployment, wherein elongating the expandable braid causes the expandable braid to move into an elongated configuration in which the expandable braid is longer and narrower than when in its expanded configuration; and
a dilator adapted to releasably hold the expandable braid in its elongated configuration.

18. The medical device of claim 17, further comprising an adhesive band disposed on the dilator, the adhesive band adapted to engage the expandable braid until axial movement of the dilator relative to the expandable braid causes the adhesive band to disengage the expandable braid.

19. The medical device of claim 17, wherein:
the dilator further comprises one or more securement features; and
the expandable braid further comprises one or more securement elements adapted to releasably engage the one or more securement features; and
the one or more securement elements are adapted to disengage with the one or more securement features in response to axial movement of the dilator relative to the expandable braid.

20. The medical device of claim 17, wherein the medical device further comprises a polymeric covering extending over the expandable braid.
